(19) United States
(12) Patent Application Publication
(10) Pub. No.: US 2011/0045077 A1
(43) Pub. Date: Feb. 24, 2011

(54) ENCAPSULATED PANCREATIC ISLET CELL PRODUCTS AND METHODS OF USE THEREOF

(76) Inventors: Gordon Weir, Cambridge, MA (US); Clark K. Colton, Newton, MA (US); Esther O'Sullivan, Dublin (IE); Amy Lewis, Cambridge, MA (US)

Correspondence Address: Pearl Cohen Zedek Latzer, LLP, 1500 Broadway, 12th Floor, New York, NY, 10036, US

(21) Appl. No.: 12/514,753

(22) PCT Filed: Nov. 13, 2007
(86) PCT No.: PCT/US07/23760

§ 371 Date: Jul. 22, 2010

Related U.S. Application Data
(60) Provisional application No. 60/858,352, filed on Nov. 13, 2006.

(51) Int. Cl. A61K 9/14; A61K 35/39; A61P 3/10; A61P 3/08; A61P 1/18

(52) U.S. Cl. 424/488; 424/484; 424/93.7

(57) ABSTRACT

This invention is directed, inter alia, to encapsulated cell products, compositions comprising the same and uses thereof to treat diabetes, and related complications, increase islet cell masses, improve a metabolic profile in a subject, and other related conditions. Processes to produce the encapsulated islet cell product are described.

### GOVERNMENT INTEREST STATEMENT

This invention was made in whole or in part with government support under Grant Numbers R01-DK50657, R01-DK063108-01A1 of the National Institutes of Health and Grant Number NCRR ICR U4Z 16606, awarded by the National Center for Research Resources of the National Institutes of Health. The government may have certain rights in the invention.

### BACKGROUND OF THE INVENTION

Diabetes is a disease that results from a person's impaired ability to produce insulin, a protein that regulates the blood glucose concentration. Insulin is produced by β cells in the Islets of Langerhans, which are aggregates of cells averaging about 150 μm in diameter and constituting about 1 to 2% of the pancreas volume. The efficacy of islet transplantation as a treatment for diabetes has been demonstrated in humans by the Edmonton Protocol, but obstacles remain for wide scale application. One major issue is that successful islet transplantation requires permanent use of multiple immunosuppressive agents. These agents may have serious side effects as well as a substantial financial burden. Microencapsulation has been used for full or partial protection of transplanted islets from immune rejection, however, the microcapsule prevents islet revascularization and creates an additional mass transfer resistance for oxygen transport to islets. This reduced oxygen transfer can lead to a hypoxic core within the islet that results in tissue death and reduced function.

Thus, it would be ideal to have an islet preparation with enhanced islet survival and function, reduced oxygen transport limitations, yet minimal immune system rejection.

### SUMMARY OF THE INVENTION

In some embodiments, this invention provides a composition comprising in vitro cultured aggregated pancreatic islet cells encapsulated in a matrix, wherein said matrix comprises a biocompatible polymer. In some embodiments, the isolated and dispersed pancreatic islet cells are cultured and aggregated in vitro. In some embodiments, aggregated pancreatic islet cells comprise beta cells.

In some embodiments, the biocompatible polymer comprises alginate. In some embodiments, a fluorocarbon is dispersed in said matrix, which in some embodiments is at a concentration of about 30 to about 85% w/v of the composition and in some embodiments, comprises perfluorodecalin.

In some embodiments, the aggregate has a diameter of about 20 to about 100 micron.

In some embodiments, the pancreatic islet cells are human in origin, and in some embodiments, the pancreatic islet cells are engineered to express a protein of interest.

In some embodiments, this invention provides a composition comprising at least one islet, islet fragment, or islet cell encapsulated in a matrix, wherein said matrix comprises a biocompatible polymer and a fluorocarbon, wherein said fluorocarbon:
i. is dispersed in said matrix; and
ii. is at a concentration of about 30 to about 85% w/v of said composition.

According to this aspect, and in some embodiments, the composition comprises a plurality of islets, islet fragments, or islet cells. According to this aspect, and in some embodiments, the biocompatible polymer comprises alginate, and in some embodiments, the fluorocarbon comprises a perfluorocarbon, which in some embodiments is perfluorodecalin.

According to this aspect, and in some embodiments, the at least one islet, islet fragment, or islet cell is human in origin, and in some embodiments, is engineered to express a protein of interest.

In some embodiments, this invention provides a method of increasing pancreatic β-cell mass in a subject, the method comprising administering to the subject a composition of this invention.

In some embodiments, this invention provides a method of altering metabolism in a subject, said method comprising administering to the subject a composition of this invention.

According to this aspect and in some embodiments, the subject is suffering from or predisposed to diabetes. In some embodiments, the method further comprises the step of administering to the subject a sulfonylurea, leptin, meglitinide, biguanide, thiazolidinedione, alpha-glucosidase inhibitor, or a combination thereof.

In some embodiments, the composition comprises pancreatic islet cells which are autologous with respect to the subject, or in some embodiments, allogeneic or in some embodiments, syngeneic or xenogeneic with respect to said subject.

In some embodiments, this invention provides a method of inhibiting, suppressing or treating diabetes in a subject, the method comprising administering to the subject a composition of this invention.

According to this aspect and in some embodiments, the subject is suffering from or predisposed to diabetes. In some embodiments, the method further comprises the step of administering to the subject a sulfonylurea, leptin, meglitinide, biguanide, thiazolidinedione, alpha-glucosidase inhibitor, or a combination thereof.

In some embodiments, the subject is insulin resistant or hypoinsulinemic, and in some embodiments, the subject suffers from maturity onset diabetes of the young (MODY).

In some embodiments, this invention provides a process for the preparation of a pancreatic islet cell product encapsulated in a biocompatible matrix comprising a fluorocarbon emulsion, said process comprising:
i. isolating and dispersing pancreatic islet cells from a pancreas of a subject;
ii. ex-vivo or in vitro culturing dispersed pancreatic islet cells obtained in (b) for a period of time sufficient to form aggregates of said pancreatic islet cells in culture; and
iii. encapsulating aggregates obtained in (b) within a matrix comprising an emulsion comprising a biocompatible polymer and a fluorocarbon.

In some embodiments, the method further comprises the step of engineering the dispersed pancreatic islet cells to express a protein of interest. In some embodiments, encapsulating comprises extrusion of said matrix through a droplet generator, and in some embodiments, capsules of about 350 μm to 3 mm in diameter are formed.

In some embodiments, this invention provides a method of increasing the viability, function, or combination thereof of insulin-secreting islets, islet fragments, aggregates or islet cells, the method comprising administering to the subject a composition of this invention. In some embodiments, the method reduces oxygen diffusion limitations in said islets, islet fragments, aggregates or islet cells, said matrix, or a combination thereof.

### BRIEF DESCRIPTION OF THE DRAWINGS

FIG. 1 is a plot of predicted oxygen profiles in 500 μm diameter microcapsules with a capsule surface P_{O2} equal to 36 mmHg. IE (islet equivalents) is a volume of tissue equal to that of a 150 μm sphere.

FIG. 2 is a plot of the predicted fractional viability and insulin secretion for an encapsulated islet or single cells with a total encapsulated volume of one islet equivalent with and without 70% (w/v) PFC Emulsion.

FIG. 3 is a plot of the predicted fraction of normal insulin secretion for a capsule that contains one islet equivalent distributed as single cells (approximated as being homogeneously distributed throughout the capsules), 50 μm aggregates, 75 μm aggregates, or 150 μm islet.

FIG. 4 is a plot of the predicted fraction of normal insulin secretion for single cells, an islet, and 50 μm aggregates at various loadings (IE=islet equivalent).

FIG. 5 is a plot of the predicted fraction of normal insulin secretion for 50 μm aggregate capsules containing 70% (w/v) PFC emulsion (IE=islet equivalent).

FIG. 6 is a micrograph of plastic sections of microcapsules stained with toluedene blue after two day culture under low oxygen conditions. (A) capsule containing an islet (B) capsule containing aggregates.

FIG. 7A is a plot of the fractional oxygen recovery of encapsulated islets and aggregates cultured for two days in 20% or 0.5% oxygen. (*=p<0.05 by student t-test). FIG. 7B is a plot of the fractional oxygen recovery of encapsulated islets in alginate with and without PFC for two days in 0.5% oxygen.

FIG. 8 is a plot of the fractional nuclei recovery of encapsulated islets and aggregates after two days of culture in 20% or 0.5% oxygen.

FIG. 9 is a plot of the insulin to DNA ratios for encapsulated islets or aggregates measured on day of encapsulation and following two days of culture in 20% or 0.5% oxygen (*=p<0.01 by student t-test).

FIG. 10 is a plot of the glucose stimulated insulin release of encapsulated islets or aggregates (Agg) in low glucose (2.8 mM) and high glucose (16.8 mM) KRHB immediately after encapsulation for the initial measurements and then after two days of culture in 20% or 0.5% oxygen.

FIG. 11 is a micrograph of paraffin sections of empty capsules transplanted into the peritoneal cavity of Lewis rats for two weeks stained with hematoxylin. (A) 1.9% (w/v) Alginate (B) 70% (w/v) PFC 0.63% (w/v) Alginate (C) 70% (w/v) PFC 0.63% (w/v) Alginate coated with PLL and Alginate.

FIG. 12 is a micrograph of plastic sections of capsules before and after two week syngeneic transplantation in non-diabetic rats. (A) islet capsules pre-transplantation, (B) aggregate capsules pre-transplantation, (C) islet capsule post-transplantation, and (D) aggregate capsules post-transplantation.

FIG. 13 is a plot of the insulin to DNA ratios measured before and after syngeneic transplantation of islet and aggregate capsules (*=p<0.01 by student t-test).

FIG. 14 is a plot of the blood glucose level of streptozotocin diabetic ICR-SCID mice following transplantation on Day 0 of varying amounts of encapsulated aggregates.

FIG. 15 is a plot of the intraperitoneal glucose tolerance test of cured recipient animals that received microcapsules containing islets or aggregates (*=p<0.01 by student t-test).

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This invention is directed, in some embodiments, to encapsulating in vitro cultured aggregates of islet cells, for example, β-cells of the pancreas in a matrix comprising a biocompatible polymer. In some embodiments, the invention is directed to the specific dispersion of a fluorocarbon, or perfluorocarbon emulsion within the matrix, which in some embodiments, enhances oxygen permeability, for example by reducing oxygen diffusion limitations in the matrix. In some embodiments, such encapsulation protects the islet derived product, for example, the aggregates, from hypoxia.

In some embodiments, this invention provides, inter alia, methods of increasing the viability, function, or combination thereof of insulin-secreting islets, islet fragments, aggregates or islet cells, the method comprising administering to the subject a composition of this invention, wherein the composition incorporates a fluorocarbon. In some embodiments, unexpectedly, the method reduces oxygen diffusion limitations in said islets, islet fragments, aggregates or islet cells, said matrix, or a combination thereof.

In some embodiments, this invention is directed to the preparation of islet cell aggregates, via dispersing pancreatic islets into single cells and allowing them to reaggregate into cell clusters. In some embodiments, the aggregates are smaller than the original islet. The smaller aggregates, in some embodiments, are less prone to the development of a necrotic core and function normally because of adequate oxygen supply. In some embodiments, the presence of cell to cell contacts in the aggregates is beneficial.

In some embodiments this invention is directed to the use of encapsulated islet cell aggregates, in a matrix comprising a biocompatible polymer. In some embodiments, the invention is directed to the use of encapsulated islet, islet fragments, aggregates and individual islet cells in microcapsules comprising a matrix comprising a biocompatible polymer, wherein a fluorocarbon is dispersed within the matrix. In some embodiments, the biocompatible polymer is alginate.

As exemplified herein, theoretical reaction-diffusion models can be developed to predict the oxygen partial pressure profile, extent of cell death, and rate of insulin secretion in microcapsules containing an islet, islet cell aggregates, and dispersed single cells exposed to specified external pO₂ values, with or without a fluorocarbon-containing compound. In some embodiments, perfluorodecalin emulsions comprising alginate were prepared, and hypoxic conditions were produced in encapsulated preparations, which comprised the perfluorocarbon (PFC). In some embodiments, alginate matrices encapsulating aggregates, surprisingly resulted in enhanced islet viability, and surprisingly, aggregate viability was markedly higher, promoting high levels of insulin secretion.

In some embodiments, modeling methods are provided, which predict that a capsule containing aggregates with half the diameter of a 150 μm islet, and a total tissue volume equivalent to one islet, can remain fully functional while the function of an intact islet has dropped to 20% of its normal level. Theoretical predictions have demonstrated that islet cell aggregates are extremely beneficial in maintaining islet cell function in low oxygen environments.

This invention provides, in some embodiments, methods to assemble various microencapsulation means for islet cells, or aggregates thereof. In some embodiments, the invention provides methods to assess the encapsulated aggregates/islets, or other tissue through nuclei counting, DNA quantification, and oxygen consumption rate measurements.

It is predicted that the enhanced capsule permeability, as a function of fluorocarbon-containing compound incorporation, encapsulating islets, islet fragments, or individual islet cells, or the encapsulation of aggregates formed from dispersed islets, promote greater islet cell survival and function, even in low oxygen environments, such as those found in encapsulated cells.

In some embodiments, this invention provides a composition comprising in vitro cultured aggregated pancreatic islet cells encapsulated in a matrix, wherein the matrix comprises a biocompatible polymer and a fluorocarbon dispersed therein. In some embodiments, the isolated and dispersed pancreatic islet cells are cultured in vitro and aggregated in culture.

Various techniques may be employed to obtain suspensions of islet cells (both differentiated and undifferentiated) from tissues, or islets, to comprise the microencapsulated preparations of this invention and/or for use in the methods of this invention. In some embodiments, isolation procedures are ones that result in as little cell death as possible. In some embodiments, the methods of isolation of pancreatic islets may be any known in the art, for example as described further herein. In some embodiments, the cells can be removed from a tissue sample by mechanical means, e.g., mechanically dispersed with a pipette. In some embodiments, cells may be dissociated from the entire tissue section, or sub-portion thereof, e.g., by enzymatic digestion of the sample, followed by isolation of the desired islet or beta cell population based on specific cellular markers, e.g., using affinity separation techniques or fluorescence activated cell sorting (FACS), or others, as will be appreciated by the skilled artisan. In some embodiments, islet cells are expanded in culture to obtain greater starting material, prior to their dissociation and/or encapsulation as described herein.

In some embodiments, the tissue is prepared using any suitable method, such as by gently teasing apart the excised tissue or by digestion of excised tissue with collagenase, via, for example, perfusion through a duct or simple incubation of, for example, teased tissue in a collagenase-containing buffer of suitable pH and tonic strength. In some embodiments, single cells are obtained, or in some embodiments, small aggregates are obtained, which in turn may be subjected to other purification techniques, such as, for example, centrifugation through Ficol gradients for concentration (and partial purification). The concentrate may in some embodiments be resuspended, into any suitable vessel, such as tissue culture glassware or plasticware, and cultured over a period of time sufficient to form the described islet cell aggregates of this invention.

In some embodiments, islets are isolated by any of the methods described herein, for example, by methods incorporated by reference, as herein described, as well as islet fragments, or individual islet cells.

In some embodiments, the term “islet fragment” is to be understood to encompass, inter alia, small sections of an islet, comprising multiple cell types, for example, alpha, beta, gamma, delta and/or PP cells. In some embodiments, the term “islet fragments” refers to a section or fragment of an islet, which may be cultured, but is not dispersed prior to its culture. In some embodiments, islet fragments are distinguished from aggregates in that aggregates represent a cell product, wherein the cells accumulate and associate in culture, following prior dispersal. In some embodiments, aggregates will comprise alpha, beta, gamma, delta and/or PP cells.

In some embodiments, digestive enzymes including proteases and DNAses are employed to disaggregate pancreatic tissue, pancreatic islets, etc., to form smaller aggregates or single cells which are then cultured in vitro for a period of time to form the aggregates of this invention.

In some embodiments, a minimum of 200,000 to about 1,000,000 cells are cultured in a 60 mm dish, to form such aggregates. In some embodiments, such counts represent the number of viable cells, for example, as obtained by prior vitality staining and counting in a haemocytometer, as will be appreciated by one skilled in the art. In some embodiments, cells are placed in cultureware treated to minimize adhesion to a surface of the flask/dish in which the cells are cultured.

In some embodiments, such ex-vivo culture prior to encapsulation will be for a time period of about 6 to about 72 hours, or about 12 to 24 hours, in some embodiments. In some embodiments, standard culture conditions of 37 degrees, 95% humidified air, 5% CO₂ are employed, and cells are cultured at about 15,000 to about 25,000, for example, 18,000 cells per cm² surface area. In some embodiments, cells are cultured at about 100,000 per ml culture medium.

In some embodiments, the aggregate will comprise about 500,000 islet equivalents of tissue, or in some embodiments, from about 200,000 to 800,000, or in some embodiments, from about 100,000 to 1,000,000. In some embodiments, a subject is administered encapsulated cell products at least once, or in some embodiments, as many times as will be necessary, which the skilled artisan will appreciate. In some embodiments, cell products/compositions as described herein may be prepared from material isolated from a subject, wherein several isolations with 1-3 infusions over several months may comprise an embodiment of a treatment regimen of this invention.

In some embodiments, aggregates obtained post in vitro culture and subsequently encapsulated will have a diameter of about 20 to about 125 micron, which in some embodiments is about 25 to about 75 micron. In some embodiments, reference to aggregate size, for example the term “aggregate diameter” reflects a value obtained as follows: light micrographs of aggregate capsules are obtained, with the area of individual aggregates within the tissue being calculated using image analysis software. The average total area of the individual aggregate was then converted to an effective diameter of a circle with equal area. Such a method was utilized and exemplified hereinbelow.

In one embodiment, tissue from which the islets/cells are derived may be adult or fetal tissue or tissue from any developmental stage. In some embodiments, the cells comprise stem and/or progenitor cells, which are differentiated in culture to form insulin-secreting cells, which are incorporated in what is considered an islet, islet fragment or islet cell, as cells which perform islet cell functions, in terms of insulin production.

In some embodiments, the source of the islet cells may be any suitable source, from any tissue in any animal or cell line, which can yield insulin secreting cells, and can be incorporated in the compositions/cell products and/or methods of this invention. For example, islet cells, islet fragments, islets or aggregates as herein described may be regenerated from stem cells (e.g. pluripotent or multipotent precursor cells) or from other starting material that can be used in islet regeneration (for example, U.S. Pat. Nos. 6,815,203, U.S. Pat. No. 7,033,831, U.S. Patent Application Publication No. 20070128176, U.S. Patent Application Publication No. 20050260749, all of which are fully incorporated by reference herein, and others, as will be appreciated by one skilled in the art). In some embodiments, the islet cells, islet fragments, islets or aggregates as herein described may be derived or isolated from any human or animal origin, including transformed cell lines, or isolated tissue, etc. In some embodiments, such islet cells, islet fragments, islets or aggregates as herein described may be administered to a subject, representing xenotransplantation as herein described. In some embodiments, any suitable animal may be utilized, for example, any rodent, such as mice or rats, porcine, canine or primate tissues or cells.

In some embodiments, cells may be differentiated in vitro, to form islet-like cells or structures, which in turn increases the pool of insulin secreting cells, and such cultures/scenarios should be considered to be part of this invention.

In some embodiments, use of less differentiated cells, as described in this aspect may provide a benefit such that the method can be practiced with relatively small amounts of starting material. Accordingly, small samples of tissue from a donor can be obtained without sacrificing or seriously injuring the donor.

In certain embodiments, the culture may be contacted with a growth factor or a composition comprising a growth factor, e.g., a mitogenic growth factor, such as, for example, IGF-I, IGF-II, or combinations thereof. In some embodiments, the composition may further comprise Exendin 4, Gastrin, Epidermal Growth Factor, or combinations thereof. In some embodiments, the compositions may comprise such growth factors, and in some embodiments, cells within the aggregates may be engineered to express or overexpress such growth factors, as will be appreciated by one skilled in the art. In some embodiments, cells within the aggregates may be engineered to express glucose transporters, which in turn may enhance glucose-sensitive insulin production in these cells. In some embodiments, such expression may be useful, for example, in the case of autologous islet cell transplant from a subject with a mutated glucose transporter, which is sub-optimally or

In some embodiments, the aggregates are formed in vitro, and in some embodiments aggregates are formed ex-vivo. The term “ex vivo” refers to cells which have been taken from a body, temporarily cultured in vitro, and then returned to body from which the cells were isolated or derived.

In some embodiments, the aggregates of this invention are specifically derived from the specific disaggregation of purified whole islets, to obtain monodispersed islet cells, or in some embodiments, suspensions with aggregates of minimal numbers of cells, which in turn further aggregate in culture.

In some embodiments, the compositions of this invention comprise ex-vivo or in vitro cultured aggregated pancreatic islet cells encapsulated in a matrix comprising a biocompatible polymer, wherein a fluorocarbon is dispersed in the matrix.

In some embodiments, this invention provides a composition comprising at least one islet, islet fragment, or islet cell encapsulated in a matrix, wherein said matrix comprises a biocompatible polymer and a fluorocarbon, wherein said fluorocarbon:
i. is dispersed in said matrix; and
ii. is at a concentration of about 50 to about 85% w/v of said composition.

According to this aspect, and in some embodiments, the composition comprises a plurality of islets, islet fragments, or islet cells. According to this aspect, and in some embodiments, the biocompatible polymer comprises alginate, and in some embodiments, the fluorocarbon comprises a perfluorocarbon, which in some embodiments is perfluorodecalin.

According to this aspect, and in some embodiments, the at least one islet, islet fragment, or islet cell is human in origin, and in some embodiments, is engineered to express a protein of interest.

In some embodiments, the biocompatible polymer comprises alginate. In some embodiments, the biocompatible polymer comprises collagen, including contracted and non-contracted collagen gels, glycosaminoglycans, hydrogels comprising, for example, but not limited to, fibrin, alginate, agarose, gelatin, hyaluronate, polyethylene glycol (PEG), dextrans, including dextrans that are suitable for chemical crosslinking, photocrosslinking, or both, albumin, polyacrylamide, polyglycolic acid, polyvinyl chloride, polyvinyl alcohol, poly(n-vinyl-2-pyrollidone), poly(2-hydroxy ethyl methacrylate), hydrophilic polyurethanes, acrylic derivatives, pluronics, such as polypropylene oxide and polyethylene oxide copolymer, or the like. In some embodiments, the biocompatible polymer comprises a fibrin or collagen, which is autologous or allogeneic with respect to the intended recipient.

In some embodiments, the fluorocarbon is at a concentration of about 30 to about 87% w/v of the composition, or in some embodiments, the fluorocarbon is at a concentration of about 30 to about 45% w/v of the composition, or in some embodiments, the fluorocarbon is at a concentration of about 40 to about 55% w/v of the composition, or in some embodiments, the fluorocarbon is at a concentration of about 50 to about 70% w/v of the composition, or in some embodiments, the fluorocarbon is at a concentration of about 60 to about 87% w/v of the composition.

In some embodiments, incorporation of a high concentration of a fluorocarbon in a composition of this invention may entail the use of certain surfactants, emulsifiers, stabilizers, excipients, etc., and other components of compositions, to achieve a stable composition, in which certain of these components may exert toxic effects on the encapsulated products therein. It is to be understood that the skilled artisan will know how to arrive at an optimum composition for a particular fluorocarbon, and other components of the composition to maximize a therapeutic effect of the compositions, while minimizing any toxic effects of the composition, but adjusting components and concentrations of the components, for example. Such adjustment is to be considered as part of this invention.

In some embodiments, the fluorocarbon comprises perfluorodecalin.

In some embodiments, the fluorocarbon may comprise perfluorooctane, perfluorodichlorooctane, perfluoro-n-octyl bromide, pertluoroheptane, perfluorodecane, perfluorocyclohexane, perfluoromorpholine, perfluorotripropylamine, perfluortributylamine, perfluorodimethylcyclohexane, perfluorotrimethylcyclohexane, perfluorodicyclohexyl ether, perfluoro-n-butyltetrahydrofuran, and compounds that are structurally similar to these compounds and are partially or fully halogenated (including at least some fluorine substituents) or partially or fully perfluorinated including perfluoroalkylated ether, polyether or crown ether. In some embodiments, the fluorocarbon may comprise a straight and/or branched chain and/or cyclic perfluorocarbons, and/or a straight and/or branched chain and/or cyclic perfluoro tertiary amines, straight and/or branched chain and/or cyclic perfluoro ethers and/or thioethers, halofluorocarbons and/or polymeric perfluoro ethers and the like.

In some embodiments, the fluorocarbon emulsion is similar to or as disclosed in U.S. Pat. Nos. 4,895,876, 4,927,623, 5,077,036, 5,114,703, 5,171,755, 5,304,325, 5,350,571, 5,393,524, and 5,403,575, fully incorporated herein by reference.

In some embodiments, the term “fluorocarbon” denotes perfluorinated or highly fluorinated carbon compounds or mixtures which are capable of transporting gases such as O₂ and CO₂. In some embodiments, the term “fluorocarbon” refers to compounds, in which most hydrogen atoms have been substituted by fluorine atoms so that a higher degree of substitution does not necessarily increase the gas transporting ability.

In some embodiments, the fluorocarbon emulsion comprises a continuous, i.e. aqueous phase and a discontinuous phase. The discontinuous phase comprises the fluorocarbon with an emulsifying agent. Osmotic agents and biological pH buffers are included, in some embodiments, in the continuous phase to maintain osmolarity and pH.

In some embodiments, the emulsifying agent surrounds and forms a layer around the discontinuous phase creating essentially fluorocarbon particles suspended within the continuous phase. In some embodiments, lecithin is used as the emulsifying agent. In some embodiments, other emulsifying agents may be used, such as fluorinated surfactants, also known as fluorosurfactants and anionic surfactants. Fluorosurfactants may comprise triperfluoroalkylcholate, perfluoroalkylcholestanol, perfluoroalkyloxymethylcholate, XMO-10 and fluorinated polyhydroxylated surfactants, such as, for examples, those discussed in “Design, Synthesis and Evaluation of Fluorocarbons and Surfactants for In Vivo Applications New Perfluoroalkylated Polyhydroxylated Surfactants” by J. G. Riess, et al Biomat. Artif. Cells Artif. Organs, 16:421-430 (1988).

In some embodiments, encapsulated cell products of this invention are prepared, or comprise some or many of the compounds/materials described in U.S. Pat. No. 5,916,790, Dionne, K. E. (1989). Effect of Hypoxia on Insulin Secretion and Viability of Pancreatic Islet Tissue. PhD, M.I.T., Cambridge, fully incorporated by reference herein.

In some embodiments, the pancreatic islet cells are human in origin, and in some embodiments, the pancreatic islet cells are engineered to express a protein of interest.

In some embodiments, this invention provides a process for the preparation of a pancreatic islet cell product encapsulated in a biocompatible matrix comprising a fluorocarbon emulsion, said process comprising:
a) isolating and dispersing pancreatic islet cells from a pancreas of a subject;
b) ex-vivo or in vitro culturing dispersed pancreatic islet cells obtained in (a) for a period of time sufficient to form aggregates of said pancreatic islet cells in culture; and
c) encapsulating aggregates obtained in (b) within a matrix comprising an emulsion comprising a biocompatible polymer and a fluorocarbon.

In some embodiments, this invention provides a pancreatic islet cell product produced by a process of this invention.

In some embodiments, the processes of this invention may make use of a technique for producing islet aggregates whereby the process forms aggregates in hanging drops, or in some embodiments, the aggregates are formed by contact with a solid substrate, for example, on a culture dish.

In some embodiments, the processes of this invention allow for high incorporation of the fluorocarbon within the encapsulated product. In some embodiments, high incorporation of fluorocarbon facilitates oxygen transport, and promotes greater aggregate islet cell viability and function.

In some embodiments, the encapsulation process and conditions employed in U.S. Pat. No. 5,912,005 or as described in Gross, J. D., Long, R., Constantinidis, I. and Sambanis, A. (2007). Biotechnol. Bioeng. 98(1), 261-270; or McGovern, K. A., Schoeniger, J. S., Wehrle, J. P., Ng, C. E. and Glickson, J. D. (2005) Magn. Reson. Med. 29(2), 196-204; or Khattak, S. F., Chin, K.-S., Bhatia, S. R. and Roberts, S.C. (2007). Biotechnol. Bioeng. 96(1), 156 (incorporated herein by reference in their entirety) are employed herein to prepare encapsulated cell products of this invention, with the products and compositions of this invention comprising materials as described therein, as will be appreciated by one skilled in the art. In some embodiments, incorporation of a fluorocarbon in the encapsulated cell products/compositions of this invention are as described in U.S. Pat. No. 5,912,005, or Zimmermann, U., Noth, U., Grohn, P., Jork, A., Ulrichs, K., Lutz, J. and Haase, A. (2000). Non-Invasive Evaluation of the Location, the Functional Integrity, and the Oxygen Supply of Implants: 19F Nuclear Magnetic Resonance Imaging of Perfluorocarbon-Loaded Ba2+− Alginate Beads. Artif. Cells Blood Substit. Immobil. Biotechnol. 28(2), 129-146, both incorporated herein by reference in their entirety.

In some embodiments, the aggregates which are encapsulated, as described herein, are prepared as described in Babensee, J. E. and Sefton, M. V. (2000). Viability of HEMA-MMA Microencapsulated Model Hepatoma Cells in Rats and the Host Response. Tissue Eng. 6(2), 165-182, incorporated by reference in its entirety.

In some embodiments, the method further comprises the step of engineering the dispersed pancreatic islet cells to express a protein of interest. In some embodiments, the protein of interest is related to or a protein expressed as part of glucose metabolism.

In some embodiments, encapsulating comprises extrusion of said matrix through a droplet generator, and in some embodiments, capsules of about 150 μm to 3 mm in diameter are formed. In some embodiments, capsules of about 300 μm to 650 μm in diameter are formed.

It will be appreciated that any method for the encapsulation of cell products as herein described, known in the art may be applied herein, and comprise aspects of this invention.

In some embodiments, this invention provides methods for treating diabetes utilizing the encapsulated islet cells or compositions comprising the same, as herein described. In some embodiments, the methods of this invention comprise administering a composition to the subject, as described, wherein the composition may comprise the encapsulated products of this invention and may optionally further comprising any therapeutic additive, including, for example, a diabetes treatment, a growth factor, a cAMP elevating agent, etc.

In another embodiment, this invention provides a method of altering metabolism in a subject, the method comprising administering encapsulated products of this invention or a composition comprising the encapsulated products of this invention and may optionally further comprise any therapeutic additive, as known to one skilled in the art.

In one embodiment, altering metabolism refers to increasing metabolism, while in another embodiment, it refers to decreasing metabolism. In one embodiment, glucose metabolism is altered. In one embodiment, the method is conducted on a subject suffering from or predisposed to diabetes.

In some embodiments, altering metabolism refers to altering, for example, improving glucose homeostasis.

In another embodiment, this invention provides a method of increasing pancreatic β-cell mass, comprising administering encapsulated products of this invention or a composition comprising the encapsulated products of this invention and may optionally further comprise any therapeutic additive, as known to one skilled in the art.

In another embodiment, this invention provides a method for the prevention or treatment of a disease associated with hyperglycemia, comprising administering encapsulated products of this invention or a composition comprising the encapsulated products of this invention and may optionally further comprise any therapeutic additive, as known to one skilled in the art.

In some embodiments, the therapeutic compound/additive which may comprise the cell products/compositions of this invention, or be utilized as part of the methods of this invention may comprise an insulin sensitivity enhancer, a glucose absorption inhibitor, a biguanide, an insulin secretion enhancer, an insulin preparation, a glucagon receptor antagonist, an insulin receptor kinase stimulant, a tripeptidyl peptidase II inhibitor, a dipeptidyl peptidase IV inhibitor, a protein tyrosine phosphatase-1B inhibitor, a glycogen phosphorylase inhibitor, a glucose-6-phosphatase inhibitor, a fructose-bisphosphatase inhibitor, a pyruvate dehydrogenase inhibitor, a hepatic gluconeogenesis inhibitor, D-chiroinsitol, a glycogen synthase kinase-3 inhibitor, glucagon-like peptide-1, a glucagon-like peptide-1 analogue, a glucagon-like peptide-1 agonist, amylin, an amylin analogue, an amylin agonist, an aldose reductase inhibitor, an advanced glycation endproducts formation inhibitor, a protein kinase C inhibitor, a γ-aminobutyric acid receptor antagonist, a sodium channel antagonist, a transcript factor NF-κB inhibitor, a lipid peroxidase inhibitor, an N-acetylated-α-linked-acid-dipeptidase inhibitor, insulin-like growth factor-I, platelet-derived growth factor, a platelet-derived growth factor analogue, epidermal growth factor, nerve growth factor, a carnitine derivative, uridine, 5-hydroxy-1-methylhidantoin, EGB-761, bimoclomol, sulodexide, Y-128, a hydroxymethylglutaryl coenzyme A reductase inhibitor, a fibric acid derivative, a β3-adrenoceptor agonist, an acyl-coenzyme A cholesterol acyltransferase inhibitor, probcol, a thyroid hormone receptor agonist, a cholesterol absorption inhibitor, a lipase inhibitor, a microsomal triglyceride transfer protein inhibitor, a lipoxygenase inhibitor, a carnitine palmitoyl-transferase inhibitor, a squalene synthase inhibitor, a low-density lipoprotein receptor enhancer, a nicotinic acid derivative, a bile acid sequestrant, a sodium/bile acid cotransporter inhibitor, a cholesterol ester transfer protein inhibitor, an appetite suppressant, an angiotensin-converting enzyme inhibitor, a neutral endopeptidase inhibitor, an angiotensin II receptor antagonist, an endothelin-converting enzyme inhibitor, an endothelin receptor antagonist, a diuretic agent, a calcium antagonist, a vasodilating antihypertensive agent, a sympathetic blocking agent, a centrally acting antihypertensive agent, an α2-adrenoceptor agonist, an antiplatelets agent, a uric acid synthesis inhibitor, a uricosuric agent a urinary alkalinizer, or any combination thereof.

In another embodiment, this invention provides a method of inhibiting, suppressing or treating diabetes in a subject, the method comprising administering encapsulated products of this invention or a composition comprising the encapsulated products of this invention and may optionally further comprising any therapeutic additive, as known to one skilled in the art.

In one embodiment, “treating” refers to both therapeutic treatment and prophylactic or preventative measures, wherein the object is to prevent or lessen the targeted pathologic condition or disorder as described hereinabove. Thus, in one embodiment, treating may include suppressing, inhibiting, preventing, treating, or a combination thereof. Thus, in one embodiment, “treating” refers inter alia to increasing time to sustained progression, expediting remission, inducing remission, augmenting remission, speeding recovery, increasing efficacy of or decreasing resistance to alternative therapeutics, or a combination thereof. In one embodiment, “suppressing” or “inhibiting”, refers inter alia to delaying the onset of associated complications or symptoms, preventing relapse to a disease, decreasing the number or frequency of relapse episodes, increasing latency between symptomatic episodes, reducing the severity of symptoms, reducing the severity of an acute episode, reducing the number of associated complications or symptoms, reducing the incidence of disease-related associated complications or symptoms, reducing the latency of symptoms, ameliorating associated complications or symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of diabetes, while in one embodiment, “secondary” refers to a symptom that is derived from or consequent to a primary cause. In one embodiment, the compounds for use in the present invention treat primary or secondary symptoms or secondary complications related to diabetes.

In another embodiment, “symptoms” may be any manifestation of a disease or pathological condition, which in one embodiment is diabetes, comprising frequent urination, excessive thirst, extreme hunger, unusual weight loss, increased fatigue, irritability, blurry vision, low insulin levels, high blood or urinary glucose levels or a combination thereof.

In one embodiment, the term “diabetes” refers to a disease of a mammalian subject, with primary, or in another embodiment, secondary diabetes, or in another embodiment, type 1 NIDDM-transient, or in another embodiment, type 1 IDDM, or in another embodiment, type 2 IDDM-transient, or in another embodiment, type 2 NIDDM, or in another embodiment, type 2 MODY, or in another embodiment, gestational diabetes, which may manifest, in some embodiments, as described, in Harrison's Internal Medicine, 14th ed. 1998.

In some embodiments, the term “diabetes” is also intended to include those individuals with hyperglycemia, including chronic hyperglycemia, hyperinsulinemia, impaired glucose homeostasis or tolerance, and insulin resistance. Plasma glucose levels in hyperglycemic individuals include, for example, glucose concentrations greater than normal as determined by reliable diagnostic indicators. Such hyperglycemic individuals are at risk or predisposed to developing overt clinical symptoms of diabetes mellitus.

In some embodiments, this invention provides methods for the treatment of diabetic complications, the method comprising administering to the subject a composition or cell product of this invention.

In some embodiments, the term “diabetic complications” refers to medical/clinical problems that occur more often in patients diagnosed with diabetes. As contemplated herein, diabetic complications include medical/clinical problems that stem from changes in blood vessels and/or nerves as a result of diabetes. These include, and are not limited to, skin conditions (i.e., bacterial infections, fungal infections, diabetic dermopathy, necrobiosis lipoidica, diabeticorum (i.e., bullosis diabeticorum), eruptive xanthomatosis, allergic skin reactions, digital scleroris, disseminated granuloma annulare, and acanthosis nigricans), gum disease, eye disorders (i.e., glaucoma, cataracts, retinopathy, kidney disease, neuropathy (i.e., systemic neuropathy, distal systemic polyneuropathy, proximal neuropathy, femoral neuropathy, neuropathic antrhropathy, cranial neuropathy, authonomic neuropathy, compression neuropathy, and diabetic amyotrophy), gout, and cardiovascular diseases/disorders (i.e., hypertension, heart disease, heart attack, stroke).

In some embodiments, the methods of this invention comprise treating a patient or subject in need. In some embodiments, the term “patient,” or “subject” describes an organism, including mammals, to which treatment with the compositions according to the present invention is provided. Mammalian species that benefit from the disclosed methods of treatment include, and are not limited to, apes, chimpanzees, orangutans, humans, monkeys; and domesticated animals (i.e., pets) such as dogs, cats, mice, rats, guinea pigs, and hamsters. In some embodiments, the cell products utilized in the compositions as described herein may be isolated/derived from any of these animals, as well.

In some embodiments, compositions of this invention may be concurrently administered to the subject, or in some embodiments, the compositions/cell products as herein described are co-administered, or concurrently administered with other compounds useful in treating diabetes, and related disorders. In some embodiments, the terms “concurrent administration” and “concurrently administering,” refer to administering at least one additional therapeutic agent suitable for use in the treatment of diabetes (i.e., insulin and/or a hypoglycemic compound), yet such administration may precede or follow administration of a cell product/composition of this invention. In some embodiments, such staggered administration may comprise a spacing of a few seconds to minutes, to days between administration of the additional therapeutic compound or compounds, and the cell product/composition of this invention. For example, at least one additional therapeutic agent can be provided in admixture with the cell product, such as in a pharmaceutical composition; or the additional therapeutic agent(s) and the cell product can be provided separately, such as, for example, separate pharmaceutical compositions administered consecutively, simultaneously, or at different times.

According to this aspect of the invention, and in one embodiment, the subject is insulin resistant and/or, in another embodiment, hypoinsulinemic. In another embodiment, the subject is predisposed to diabetes.

In another embodiment, the methods of this invention may further comprise the step of administering to the subject an additional diabetes medication, as part of a combination therapy. In one embodiment, the diabetes medication may comprise a sulfonylurea, leptin, meglitinide, biguanide, thiazolidinedione, alpha-glucosidase inhibitor, or a combination thereof.

In another embodiment, the methods of this invention may further comprise administering to the subject, or in another embodiment, contacting cells in the subject, with a GLP-1 receptor agonist.

In one embodiment, the GLP-1 agonist may include naturally occurring peptides such as GLP-1, exendin-3, and exendin-4 (see, e.g., U.S. Pat. No. 5,424,286; U.S. Pat. No. 5,705,483, U.S. Pat. No. 5,977,071; U.S. Pat. No. 5,670,360; U.S. Pat. No. 5,614,492), GLP-1 analogs or variants (see, for example, U.S. Pat. No. 5,545,618 and U.S. Pat. No. 5,981,488), and small molecule analogs. GLP-1 receptor agonists may be tested for activity as described in U.S. Pat. No. 5,981,488.

It should be understood that in addition to the ingredients particularly mentioned above the formulations of this invention may include other agents or excipients conventional in the art having regard to the type of formulation in question, for example those suitable for oral administration may include flavoring agents.

It will be apparent to those skilled in the art that various modifications and variations can be made in the compositions, use and preparations of the present invention without departing from the spirit or scope of the invention.

For administration to mammals, and particularly humans, it is expected that in the case of medications, the physician or other qualified healthcare provider may determine the actual dosage and duration of treatment, which will be most suitable for an individual and can vary with the age, weight and response of the particular individual. It will be appreciated that in the case of non-prescription (e.g. “over-the-counter”) medications, foods, food products, food supplements, cosmetic and personal care compositions, the amount may be determined at the discretion of the user, optionally with guidance from the labeling or from an appropriate health care provider or other advisor.

In some embodiments, the compositions/cell products of this invention are administered in an effective amount to treat the described disease or condition. In some embodiments, the term “effective amount” or “therapeutic effective amount,” refers to the amount necessary to elicit the desired biological response. In accordance with the subject invention, the therapeutic effective amount is the amount of a compositions/cell products of this invention and optionally at least one additional therapeutic agent necessary to treat and/or ameliorate diabetes as well as decrease the severity or prevent a particular diabetes-related complication (i.e., retinopathy, glaucoma, cataracts, heart disease, stroke, hypertension, neuropathy, dermopathy, gum disease, etc.). The decrease may be a 10%, 20%, 30%, 40%, 50%, 60%, 70%, 80%, 90%, 95%, 98% or 99% decrease in severity of disease and/or complications related thereto.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or subrange within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

In the claims articles such as “a,”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

### EXAMPLES

### Materials and Methods

### Animals:

Male Lewis or Sprague-Dawley rats weighing 200-250 g were used as islet donors. Lewis rats were used as recipients for syngeneic and empty capsule transplant experiments. Hyperglycemic ICR-SCID mice were used as diabetic recipients. Diabetes was induced with an intraperitoneal injection of streptozotocin at 180 mg/kg 10 to 14 days prior to transplantation. Mice having fed glucose levels higher than 350 mg/dl in the morning on two separate occasions were used as recipients. Normoglycemia was defined as two consecutive measurements on different days with non-fasting blood glucose levels below 200 mg/dl. Animals were kept in a conventional animal facility with free access to food and water.

### Islet Isolation:

Rat islets were isolated according to standard techniques. Briefly a laparotomy is performed under anesthesia with Ketamine/Xylazine. The common bile duct was cannulated and 8-10 ml of rodent liberase R10.33 mg/ml in minimum essential media (MEM) containing 100 mg/dl glucose was injected. The animal was then exsanguinated; the inflated pancreas was then removed and incubated in a circulating water bath at 37° C. for 24.5 minutes. The digested pancreatic tissue was washed with MEM containing 10% newborn calf serum and then strained through 400 μm wire mesh. Islets were purified by centrifugation at 1750×g for 17 minutes through a discontinuous hisotpaque 1077 gradient. Islets of 50-250 μm in diameter were handpicked under a dissecting microscope, counted, and then cultured overnight in islet culture media at 37° C. in a humidified air atmosphere containing 5% CO₂. Islet culture media is RPMI 1640 with glucose 100 mg/dl supplemented with 10% fetal calf serum, penicillin-streptomycin (10,000 units/ml and 10,000 μg/ml, respectively), glutamine (2 mmol/1), and HEPES (238 mg/ml).

### Islet Dispersion and Reaggregation:

Islets were washed in PBS-calcium and magnesium free. They were then centrifuged and the supernatant was removed. 5 ml of a solution of Bovine pancreas trypsin (Sigma Aldrich) 1 mg/ml and DNAse 30 ug/ml in PBS was added per 4000 islets. The islet suspension was incubated at 37° C. for 13.5 minutes vortexing every 4.5 minutes. Cold islet culture media was added to stop the digestion. Cells were counted using a haemocytometer, and then put in ultra low attachment dishes (Corning) at 500,000 cells per 60 mm dish. They were cultured overnight prior to encapsulation.

### Microencapsulation:

Microcapsules were produced using purified alginate with high guluronic acid content (SLG 100, FMC Polymer, Norway) at a concentration of 1.5% w/v in 0.9% w/v NaCl. Whole islets or islet cell aggregates were added to form a tissue-alginate suspension. Microcapsules were produced by extrusion of the tissue-alginate suspension through an electrostatic droplet generator (Pronova Polymer, Norway) into 20 mM BaCl₂ solution. Microcapsules from different batches typically ranged from 350 μm to 600 μm in diameter with an average for each batch between 400 μm and 500 μm. After sequential washes in Hepes buffer and Krebs buffer the microcapsules were placed into culture.

### Encapsulated Tissue Culture:

Capsules that were cultured in 20% oxygen were placed in standard polystyrene culture flasks in islet culture media at a density at which there were no oxygen limitations, and placed in a standard humidified air incubator with 5% CO₂ at 37° C. Capsules to be cultured at 0.5% oxygen were placed on silicone rubber bottom dishes in islet culture media and then placed into a humidified incubator where the gas levels were controlled to be at 0.5% O₂ and 5% CO₂ with balance N₂. Capsules were cultured for two days under these conditions for the in vitro experiments.

PFC Alginate Preparation: One method to enhance the permeability of alginate microcapsules for islet transplantation is to make the capsules from an alginate solution that contains a perfluorocarbon (PFC) emulsion. Perfluorocarbons are highly desirable materials for enhancing oxygen delivery due to their very high oxygen solubility, approximately 25 times that of water on a volumetric basis. Enhanced solubility will lead to enhanced permeability because the permeability is the product of the gas solubility and diffusivity in the material of interest. A perfluorocarbon emulsion made from perfluorodecalin 70% (w/v) and 20% (w/v) Intralipid® (Baxter), (a soybean oil emulsion with composition 20% (w/v) soybean oil, 1.2% (w/v) egg yolk phospholipid, 2.3% (w/v) glycerin with balance water) was prepared (Schweighardt and Kayhart 1990). Alginate can be dissolved directly into the PFC emulsion at a concentration of 0.63% (w/v) alginate and then capsules were made using the standard encapsulation technique previously described. The PFC concentration in the microcapsule is 70% (w/v).

### Oxygen Consumption Rate (OCR):

Oxygen consumption rates were measured in DMEM without serum by sealing tissue microcapsule suspensions in a 200 μl stirred titanium chamber maintained (Instech Laboratories) at 37° C. The time dependent PO₂ within the chamber was recorded with a fluorescence-based oxygen sensor (Ocean Optics), and the data at high PO₂ was fit to a straight line. The maximal OCR was evaluated from OCR=V_{ch}α(ΔPO₂/Δt), where V_{ch} is the chamber volume and α is the Bunsen solubility coefficient for the contents of the OCR chamber.

### Capsule Dissolution:

Many of the quantitative measures of encapsulated tissue-nuclei counts, DNA content, alginate content, or insulin content require that the capsule first be dissolved. Tissue was removed from microcapsules by incubating the capsules in 800 μl of 100 mM tetrasodium EDTA (pH=8) for 1 hr at 37° C. with vortex mixing every 30 minutes.

### Nuclei Counts:

Aliquots of 100 μl of islet tissue from dissolved capsules (˜2.5×10⁶ cells/ml or 125 IEQ/ml) were incubated with a lysis solution. Cells were disrupted by incubation of aliquots of 100 μl of islet tissue from dissolved capsules (˜2.5×10⁶ cells/ml or 125 IEQ/ml) with a lysis solution and shearing through a needle. The liberated nuclei were stained with 7-Aminoactinomycin-D (7-AAD) at a final concentration of 0.01 mg/ml and counted using a flow cytometer (Guava PCA).

### DNA Analysis:

DNA concentration was quantified by fluorospectrophotometry using the CyQUANT® Cell Proliferation Assay Kit (Molecular Probes), which is based on the strong fluorescence enhancement that the CyQUANT GR dye undergoes when bound to cellular nucleic acids. Fluorescence resulting from CyQUANT dye binding to RNA was eliminated by pre-treating the samples with DNAse-free RNAse. The fluorescence intensity was linearly related to the concentration of nucleic acids in the sample.

Insulin and DNA content: An aliquot of the islet or islet cell aggregate suspension in EDTA was frozen, then thawed and diluted to a known volume with PBS-cmf. The samples were sonicated on ice. An aliquot was taken for insulin immediately adding 2× high salt buffer (NaCl 2.15M, NaH₂PO₄ 0.01M, Na₂HPO₄ 0.04M, EDTA 0.002M) and then transferring to 4° C. for overnight insulin extraction. Thereafter the sample was stored at −20° C. until assayed. Insulin content was determined using a rat insulin Radioimmunoassay kit (Linco, St Charles, Mo.) or rat insulin ELISA kit (ALPCO diagnostics, Salem, N.H.) after appropriate dilution.

### Alginate Content:

The alginate content of samples were assayed using DMMB (dimethyl methylene blue) dye which is a cation that binds to polyanions such as alginate by a method adapted from the literature (Halle et al. 1993). Upon binding the absorbance maximum shifted from 650 nm to 520 nm. The ratio of the absorbance of a sample at the wavelengths of 520 nm and 650 nm increased linearly with increases in alginate concentration.

### Glucose Stimulated Insulin Release:

Encapsulated islets or islet cell aggregates suspended in Krebs Ringer Hepes buffer (KRHB) (NaCl 137 mM, KCl 4.8 mM, KH₂PO₄ 1.2 mM, MgSO₄.7H₂0 1.2 mM, CaCl₂.2H₂0 2.5 mM, NaHCO₃ 5 mM, HEPES 16 mM, BSA 0.1% (w/v), pH 7.4) containing 2.8 mM glucose. Five 1 ml aliquots with approximately equal numbers of microcapsules containing islets or islet cell aggregates were pre-incubated at 37° C. in humidified air and 5% CO₂ for one hour. The in vitro insulin secretion was then assessed by consecutive incubations of one hour in KRBH containing 2.8 mM glucose and 16.8 mM glucose. At the end of each incubation 800 μl of the KRBH was removed and frozen for insulin determination by ELISA (ALPCO diagnostics, Salem, N.H.).

### Histology:

Encapsulated islets or islet cell aggregates were fixed in 2.5% w/v glutaraldehyde. Plastic 1 μm thick sections were made and stained with toluedene blue.

Transplantation of Microcapsules into the Peritoneal Cavity:

Microcapsules were injected into the peritoneal cavity of recipient rats or mice through a central midline 5-10 mm incision using a sterile plastic transfer pipette. The incision was closed using 5/0 silk, the outer layers were stapled. The animals were under anesthesia with Ketamine/Xylazine.

### Intraperitoneal Glucose Tolerance Test:

Transplant recipients were fasted overnight for 15 hours. They received 2 g/kg of glucose by intraperioneal injection using a 10% (w/v) glucose solution. Blood glucose was measured immediately prior to the injection (time 0) and at 15, 30, 60, 90 and 120 minutes thereafter. Blood was obtained from the tail vein and applied to a glucometer (One Touch).

### Statistics:

Data are expressed as average±standard deviation. Statistical significance was determined by a two-way Student t-test.

### Example 1

### Predictive Oxygen Profiles in Microcapsules Comprising Single Cells, Aggregates, or Islets

To assess the benefits of aggregate and PFC-containing microcapsules, a theoretical model was developed to predict the local partial pressure of oxygen, which, in turn, was used to assess tissue viability and fraction of normal insulin secretion for encapsulated islets, aggregates, and single cells. Predictions of the model were with respect to a 500 μm capsule.

A three-dimensional species conservation equation was utilized for reaction and diffusion to predict the oxygen profile within the microcapsule:

Dᵢ∇²Cᵢ=Vᵢ Eq. (1)

where Dᵢ (cm²/s) is the effective diffusivity of oxygen in subdomain i, Cᵢ (mol/cm³) is the concentration of oxygen in subdomain i, and Vᵢ (mol/cm³/s) is the local oxygen consumption rate per unit volume in subdomain i. For convenience, since partial pressures are equal across interfaces of different materials, we make use of oxygen partial pressure instead of concentration, which are related by

C=αP Eq. (2)

where α (mol/cm³/mmHg) is the effective Bunsen solubility coefficient in subdomain i and P is the partial pressure of oxygen. Combining Eq. (1) and Eq. (2) gives

(αD)ᵢ∇²Pᵢ=Vᵢ Eq. (3)

The oxygen consumption rate is assumed to follow Michaelis-Menten kinetics for all tissue,

$${V}_{i} = \frac{{V}_{max} {ɛ}_{i} {P}_{i}}{{K}_{m} + {P}_{i}} \qquad Eq . ( 4 )$$

where Vₘₐₓ is the maximum oxygen consumption rate for the tissue, εᵢ is the tissue volume fraction in subdomain i, and Kₘ is the Michaelis-Menten constant. For the capsule that contains an islet or aggregates, no oxygen is consumed in the alginate subdomain; therefore Vᵢ is equal to zero, and the tissue volume fraction in the islet or aggregate subdomains is equal to one. For the case of a capsule containing dispersed single cells that have a total volume equal to a 150-μm islet (an islet equivalent), the model consists only of one subdomain, and the tissue volume fraction in a 500-μm capsule (e) is equal to 0.027.

Eq. (3) is solved simultaneously for all subdomains of the microcapsule subject to the following conditions. The islet is assumed to be centrally located. At the capsule center a symmetry boundary condition is used. At the boundary between a tissue subdomain and the alginate subdomain the partial pressure of oxygen and the flux of oxygen across the boundary are both equal. The final boundary condition that is required to solve the equations is the assumption that the external partial pressure of oxygen is specified at the capsule surface (P_{S}). The capsules containing aggregate have spheres of tissue of 50-, or 75-μm in diameter arranged in a cubic array at varying packing densities.

The relationship used to predict the local fraction (F_{S}) of normal insulin secretion rate as a function of local oxygen partial pressure within the islet was developed from data on the effect of hypoxia on islet insulin secretion, estimating the oxygen partial pressure profile within the islet in the experimental system, and then determining the simplest model type and parameters(s) that best predict the insulin secretion level (Dionne et al. (1993). Diabetes. 42, 12-21.; Avgoustiniatos, E. S. (2001). Oxygen Diffusion Limitations in Pancreatic Islet Culture and Immunoisolation. PhD Thesis, M.I.T., Cambridge, Mass.). The result for a value of Vₘₐₓ=4×10⁻⁸ mol/cm³/s is

$$\begin{matrix}P < 5.1 mmHg & {F}_{s} = \frac{P}{5.1} \\ P \geq 5.1 mmHg & {F}_{s} = 1.0\end{matrix} \qquad Eq . ( 5 )$$

The fraction of normal insulin secretion averaged over all tissue within the microcapsule was determined by evaluating the volume integral of Fₛ in all tissue containing sub-domains. The fractional viability of the encapsulated tissue was estimated by determining the volume fraction of tissue where P>P_{C}. P_{C} is the critical oxygen partial pressure below which tissue dies and the value of P_{C} is assumed to be 0.1 mmHg.

Oxygen profiles were calculated for a centrally located 150-μm diameter islet with and without PFC emulsion in the microcapsule and for a capsule containing many 50-μm diameter aggregates with a total tissue volume of 1.2 islet equivalents at a capsule surface P_{O2} of 36 mmHg (FIG. 1). Comparing the oxygen profiles for the capsules containing a single centrally located intact islet, the oxygen level at the outer surface of the islet is higher when PFC emulsion is incorporated into the alginate phase of the microcapsule. Comparing the oxygen profile in a normal alginate capsule that contains an islet or aggregates, the minimum P(O₂) that the tissue experiences in the microcapsule is significantly increased for the aggregates (Pₘᵢₙ=26 mmHg) compared to the islet where the P(O₂) is sufficiently reduced, causing tissue death (Pₘᵢₙ<0.1 mmHg). The minimum oxygen level experienced by tissue in capsules containing aggregates that also contain PFC emulsion is even further increased (Pₘᵢₙ=30 mmHg).

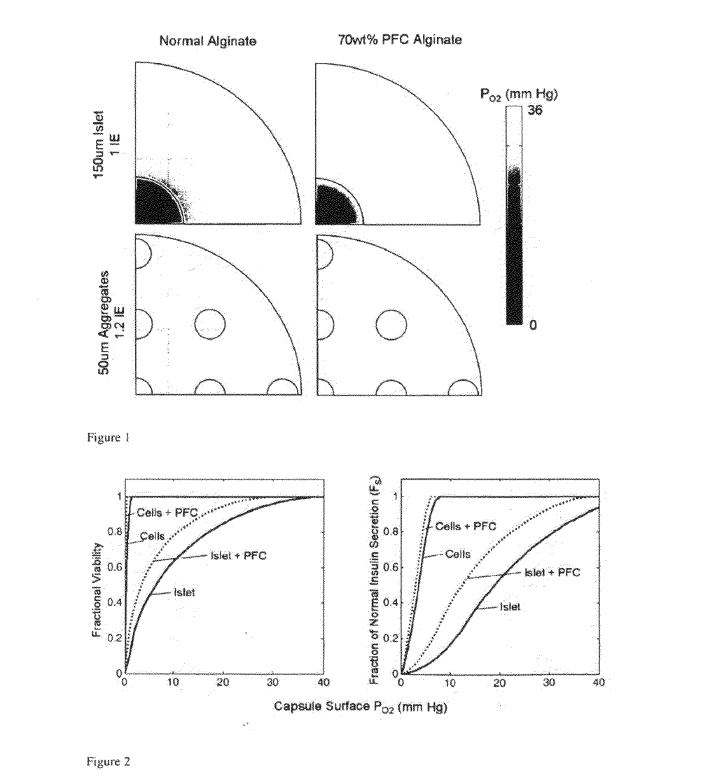

Oxygen profiles were calculated for a variety of tissue configurations and capsule surface oxygen levels in order to predict viability and fraction of normal insulin secretion. The predictions allowed assessment of the benefits of both methods of enhancing oxygen delivery to microencapsulated tissue (smaller islet aggregates and incorporating PFC emulsion into the microcapsule). The first comparisons of tissue viability and insulin secretion were for a centrally located intact islet or one islet equivalent of tissue homogenously distributed throughout the entire capsule to approximate the case of encapsulated single cells (FIG. 2). The homogenous case represents the limit of the best case scenario for tissue distribution in a microcapsule; both tissue viability and insulin secretion are maintained in lower oxygen environments in capsules containing single cells. Adding 70% (w/v) PFC emulsion to the single cell capsules has a minimal effect. Adding PFC emulsion to microcapsules containing islets results in a modest improvement in the oxygen environments where intact islets maintain viability and insulin secretion.

Calculations were also performed for microcapsules comprising varying concentrations of PFC for example at 110, 90, 70 and 30% (w/v) of the capsule, which showed beneficial effects for all concentrations tested.

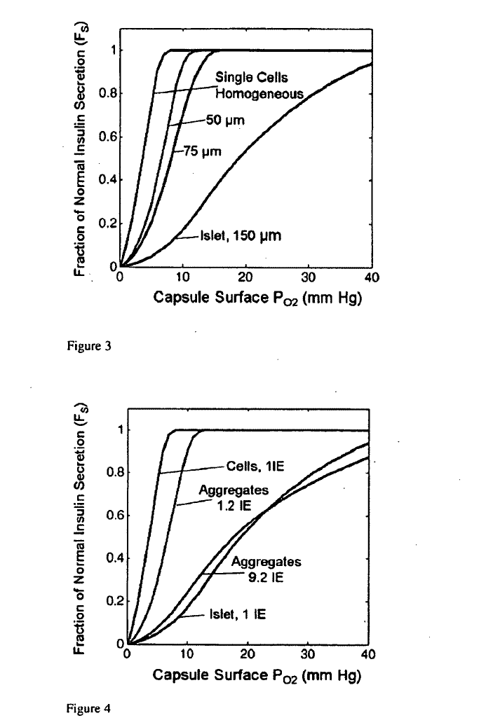

The next comparison that was made was to examine the effect of breaking up a 150-μm islet into 50- or 75-μm diameter aggregates and comparing microcapsules that all have a total tissue volume equal to one islet equivalent (FIG. 3). The predictions for normal insulin secretion show that the use of aggregates of either size allows for the tissue to secrete insulin normally in much lower oxygen environments than an islet and are actually quite similar to the single cell case.

The predictions for fraction of normal insulin secretion for single cells (1 islet equivalent) and an intact islet (1 islet equivalent) were compared to 50-μm aggregates with a total capsule tissue loading of 1.2 or 9.2 islet equivalents (FIG. 4). For the case of 1.2 islet equivalents of aggregate tissue, function was maintained at lower oxygen than an intact islet. Increasing the capsule tissue loading to 9.2 islet equivalents of aggregates, the predicted fraction of normal secretion was similar to one intact islet over the range of oxygen levels examined. Much higher tissue loadings of capsules can be achieved for aggregates as compared to islets which can result in reduced total transplant volumes. It is advantageous to reduce the total transplant volume as the large volume of microcapsules required to treat diabetic patients is a drawback for the use of microcapsules.

Predictions for fraction of normal insulin secretion were also made for microcapsules containing 50-μm aggregates with a total tissue volume of 1.2 or 9.2 islet equivalents with 70% (w/v) PFC emulsion incorporated into the capsule (FIG. 5). Increasing the oxygen permeability of the alginate phase results in marginal improvements in insulin secretion for capsules with aggregate loadings of 1.2 islet equivalents. However, incorporating PFC into capsules at high tissue loadings (9.2 islet equivalents) offers an improvement in the ability for the tissue to function in lower oxygen environments.

Calculated oxygen profiles in microcapsules for capsules containing single cells, aggregates, and islets were obtained. The oxygen profiles were then used to predict tissue fractional viability and fraction of normal insulin secretion. The model predictions demonstrated that using smaller aggregates as opposed to intact islets greatly improved the ability of encapsulated tissue to function in lower oxygen environments. Incorporating a PFC emulsion into the microcapsule, to increase the material's permeability to oxygen, allowed for intact islets to function in lower oxygen and was beneficial to capsules containing aggregates at high tissue loadings.

### Example 2

### In Vitro Characterization of Microcapsules Comprising Single Cells, Aggregates, or Islets

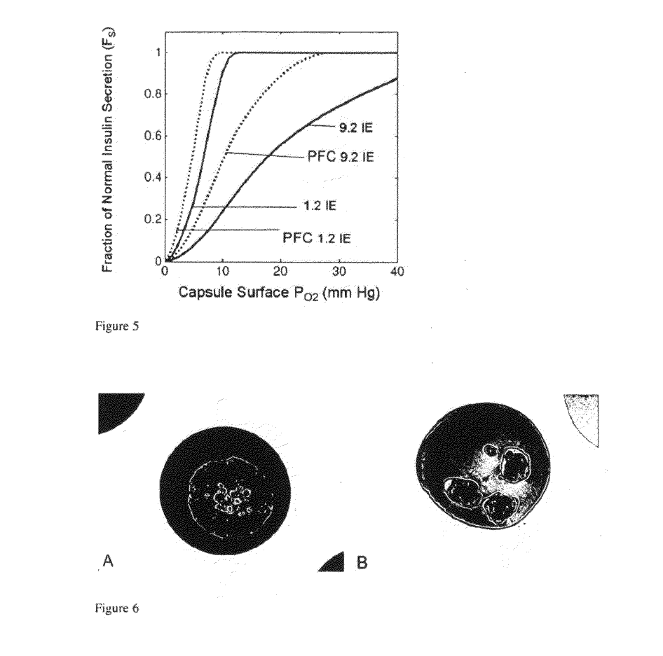

Histology sections were prepared to examine encapsulated islets or aggregates following culture under low oxygen conditions. FIG. 6A shows an encapsulated islet after 2 days of culture under low oxygen conditions where cells in the center or the islet were only stained lightly by toluedene blue indicating that the cells at the center of the islet were necrotic due to a lack of oxygen. In marked contrast, FIG. 6B shows a section of a microcapsule containing aggregates after 2 day culture under low oxygen conditions where the tissue was healthy without signs of necrosis. Qualitative observations of encapsulated tissue histological sections indicated that encapsulated aggregates survived better than encapsulated islets in low oxygen.

Oxygen consumption rate measurements of encapsulated tissue were performed on the day of encapsulation and again after two days of culture under normal (20%) or low (0.5%) oxygen conditions. OCR recovery was calculated using the following equation:

$$Fractional OCR Recovery = \frac{{( OCR per ml capsule )}_{After 2 Day Culture}}{{( OCR per ml capsule )}_{After Encapsulation}} \qquad Eq . ( 6 )$$

The OCR was measured for a sample of capsules. The capsules were removed from the chamber and dissolved by the previously described method for capsule dissolution. The dissolved capsule tissue and alginate suspension were analyzed to determine the insulin content, nuclei count, DNA content, or alginate content of the sample within the OCR chamber. The alginate content was measured in order to normalize the OCR results by the volume of capsules in the chamber. The capsule volume normalization was necessary due to the high variability of capsule sampling.

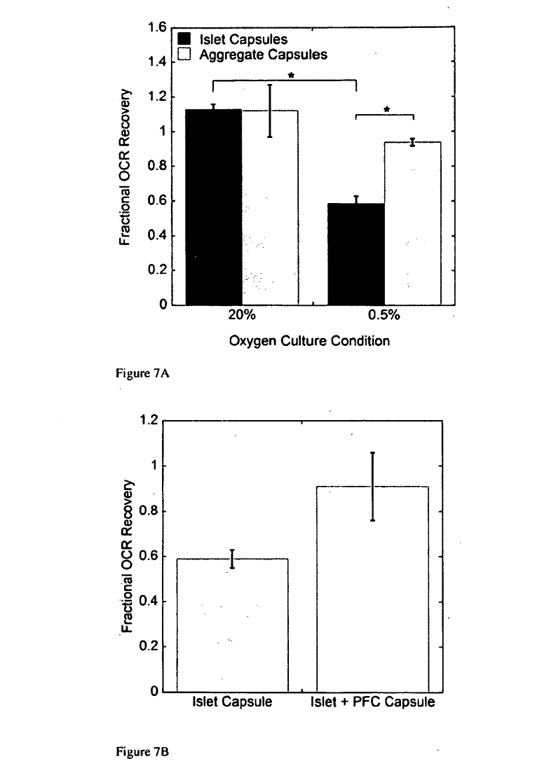

The fractional OCR recovery results were plotted as depicted in FIG. 7A. Under normal culture conditions (20% O₂) all of the viable tissue was recovered for both aggregate and islet capsules as the measured fractional OCR recovery was greater than one. Culture in low oxygen (0.5%) resulted in recovery of almost all viable tissue in aggregate capsules (fractional OCR Recovery=0.94) but significantly less viable tissue from the islet capsules (fractional OCR Recovery=0.59). The measured fractional OCR recoveries demonstrated that encapsulated aggregate survival was better in low oxygen than encapsulated islet survival.

OCR recovery was measured for capsules containing islets with or without PFC emulsion cultured for two days in low (0.5%) oxygen (FIG. 7B). The OCR recovery in low oxygen for capsules containing islets and PFC was 0.91 indicating reduced oxygen limitations compared to an oxygen recovery of 0.59 for the capsules containing islets without PFC. While histological examination of tissue in PFC emulsion capsules showed some toxicity effect, the skilled artisan will appreciate that an optimal value for PFC incorporation to maximally produce the desired effect, and concurrently minimally result in toxicity may be arrived at, for example, by electing suitable excipients, diluents, detergents, etc., in the compositions.

The tissue recovery from culture in normal (20%) and low (0.5%) oxygen was also determined from the dissolved capsule sample.

$$Fractional Nuclei Recovery = \frac{{( Nuclei per ml capsule )}_{After 2 Day Culture}}{{( Nuclei per ml capsule )}_{After Encapsulation}} \qquad Eq . ( 7 )$$

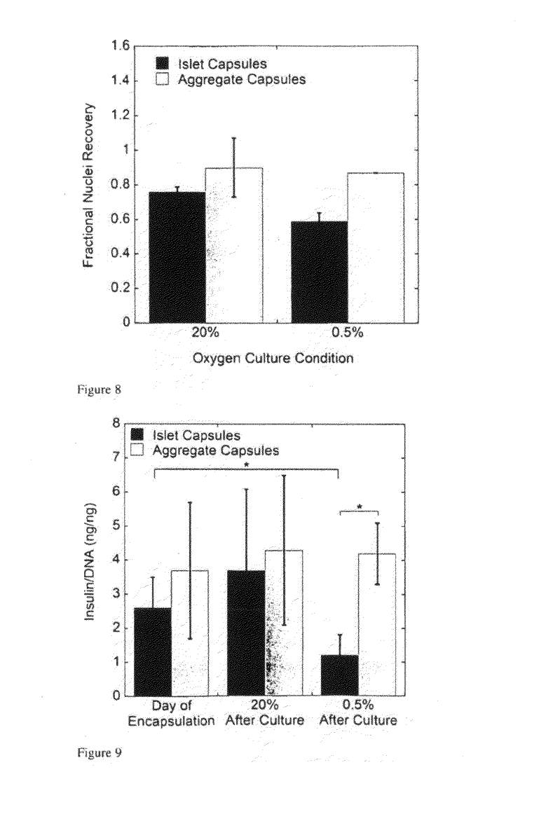

Again the alginate content of the sample was measured to normalize the nuclei count by the total volume of capsules in the sample due to the high variability of capsule sampling. The fractional nuclei recoveries after two days of culture in normal and low oxygen were plotted as depicted in FIG. 8. The data shows that under both culture conditions the tissue recovery from aggregate capsules was higher than for islet capsules. The tissue recovery is the lowest for islet capsules cultured at 0.5% oxygen. Disagreement between total tissue loss values and viable tissue loss may be a reflection of the short experimental time thus non-viable was not yet cleared from the capsule.

Comparison of the insulin content to the DNA content of an islet sample was another measure of tissue viability. Insulin and DNA measurements were performed on encapsulated islets or aggregates on the day of encapsulation or after 2 days of culture in 20% or 0.5% oxygen (FIG. 9). For all conditions, the insulin to DNA ratio was higher for the aggregate capsules compared to the islet capsules and significantly higher for the capsules cultured in 0.5% oxygen. It was also observed that the insulin to DNA ratio was significantly decreased for the islet capsules after 2 days of culture in 0.5% oxygen compared to the islet capsules on the day of encapsulation. The measured insulin to DNA ratios showed that a viability loss for encapsulated islets cultured in low oxygen existed while the encapsulated aggregate viability was maintained.

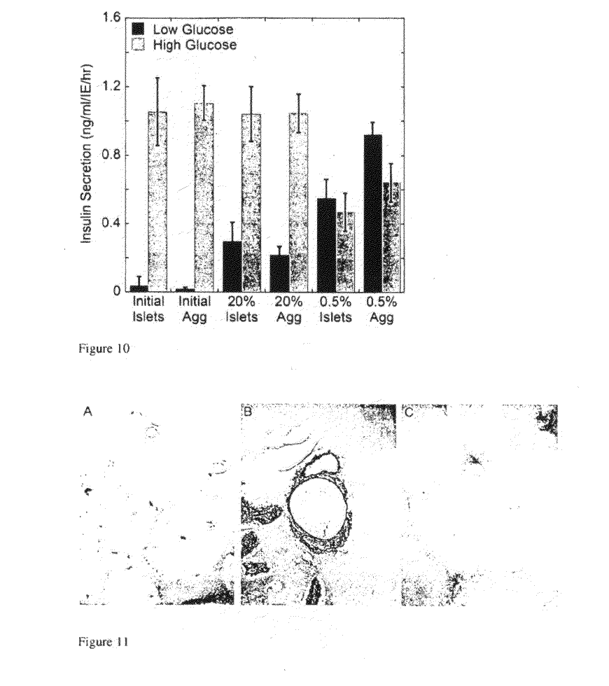

The ability of encapsulated islets or aggregates to secrete insulin in response to low (2.8 mM) or high (16.8 mM) glucose was assessed for tissue on the day of encapsulation and then after two days of culture in normal (20%) or low (0.5%) oxygen (FIG. 10). After encapsulation and following two days of culture both aggregate and islet tissue were capable of secreting more insulin in response to high glucose. However, after culture in low oxygen the islets did not secrete more insulin in response to high glucose. The lack of an increase in glucose-stimulated insulin release was to be expected as 0.5% oxygen is equivalent to a P_{O2} of 3.5 mmHg which is in the range where the insulin secretion model predicted insulin secretion would be disrupted. 3.5 mmHg corresponds to the P_{O2} in the gas phase and the tissue within the microcapsule is exposed to even lower oxygen levels.

### Example 3

### Encapsulated Aggregate Transplantation Reverses Diabetic Phenotype

Empty alginate capsules of three types: (1) no PFC emulsion 1.9 wt % alginate, (2) PFC emulsion 0.63 wt % alginate, and (3) PFC emulsion 0.63 wt % alginate coated with poly-L-lysine and alginate were transplanted into the peritoneal cavity of Lewis rats for 2 weeks. The PFC emulsion that was incorporated into the microcapsules was 70 wt % (w/v) perfluorodecalin. At two weeks the animals were sacrificed and the capsules were recovered from the peritoneal cavity. Capsules were embedded in agar, embedded in paraffin, sectioned, and stained with hematoxylin to examine whether or not they elicited an immune reaction. Alginate capsules that did not contain PFC emulsion caused no immune response in vivo as no cells were detected attached to the capsule surface after retrieval (FIG. 11A). PFC emulsion containing capsules with no additional coatings elicited a strong immune reaction as can be seen in the histological sections where the capsules are covered with many layers of cells on the outside (FIG. 11B). PFC emulsion containing capsules were biocompatible when a poly-L-lysine and alginate coating was applied to the outside as again no cells were attached to the outside of the capsules after transplantation (FIG. 11C).

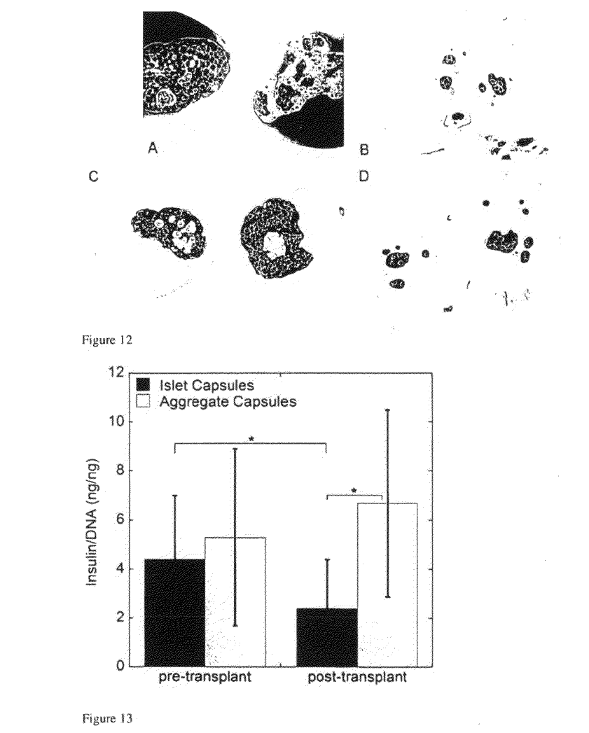

Syngeneic transplants of microencapsulated Lewis rat islets or aggregates into non-diabetic animals were performed to assess tissue survival under transplant conditions where there is no immune response or hyperglycemia. Histology sections of the tissue before and after transplantation are shown in FIG. 12. The islet capsules prior to transplantation showed no evidence of central necrosis however some cells were faintly stained indicating that the islet was not completely healthy (FIG. 12A). Following transplantation the capsules containing islets showed clear indication of central necrosis due to oxygen limitations at the islet core and complete absence of cells at center of one of the encapsulated islets (FIG. 12C). Aggregate capsules before and after transplant appeared healthy and no central necrosis was observed (FIGS. 12 B&D).

Insulin to DNA ratios were measured for islet and capsules containing aggregates before and after transplant (FIG. 13). Before transplant the insulin to DNA ratios were quite similar for encapsulated aggregates and islets. After transplantation the insulin to DNA ratio of encapsulated aggregates was significantly higher as compared to encapsulated islets. There is also a significant drop in the insulin to DNA ratio for the encapsulated islets after transplantation as compared to the pre-transplantation sample. The insulin to DNA measurements indicated that aggregates survived better after being transplanted within microcapsules into the peritoneal cavity as compared to islets transplanted under the same conditions.

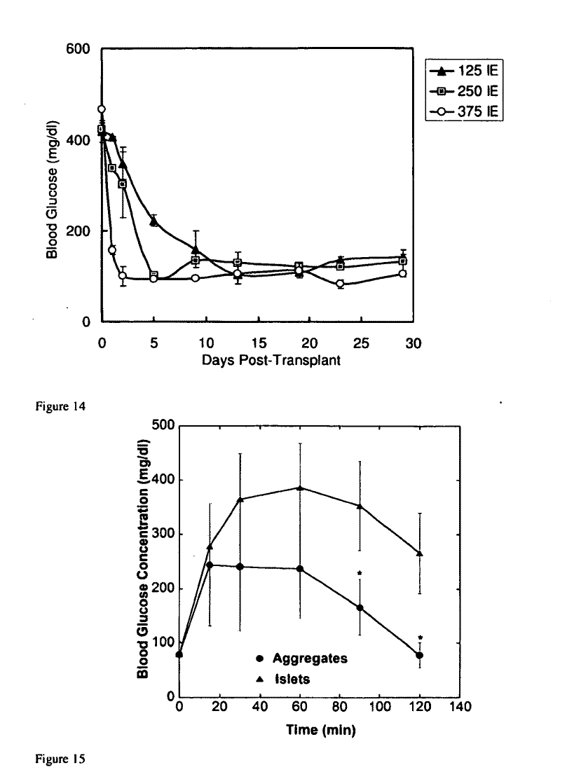

Varying amounts of encapsulated rat aggregates were transplanted into the peritoneal cavity of streptozotocin diabetic immunocompromised ICR-SCID mice. The blood glucose level of all animals was measured over the course of 30 days following transplantation (FIG. 14). The amount of tissue that was to be transplanted was measured by nuclei counting. All animals transplanted with 375, 250, or 125 islet equivalents were cured (blood glucose<200 mg/dl) and remained non-diabetic for the length of the experiment (30 days). Aggregate-containing microcapsules were able to reverse diabetes in streptozotocin diabetic mice with as few as 125 islet equivalents of transplanted tissue.

Capsules containing 125 islet equivalents (IE) of islets or aggregates prepared from islet isolations were transplanted intraperitoneally to hyperglycemic ICR-SCID mice. Normoglycemia was achieved in 75% of aggregate-containing capsule recipients and 77% of islet-containing capsule recipients (p>0.05). At 56 days post-transplantation intraperitoneal glucose tolerance tests were performed. Results showed significantly lower blood glucose values in the aggregate-containing capsule recipients (n=3) compared to the islet-containing capsule recipients (n=6) at time 90 and 120 min after glucose injection as shown in Error! Reference source not found. This indicated better glucose tolerance in the animals that received capsules containing aggregates.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising in vitro cultured aggregated islet cells or precursors thereof encapsulated in a matrix, wherein said matrix comprises a biocompatible polymer.

2. The composition of claim 1, wherein isolated and dispersed islet cells or precursors thereof are cultured in vitro to form said aggregated islet cells or precursors thereof.

3. The composition of claim 1, wherein said aggregated islet cells comprise beta cells.

4. The composition of claim 1, wherein said biocompatible polymer comprises alginate.

5. The composition of claim 1, wherein a fluorocarbon is dispersed in said matrix.

6. The composition of claim 5, wherein said fluorocarbon is at a concentration of about 40 to about 85% w/v of said composition.

7. The composition of claim 5, wherein said fluorocarbon comprises perfluorodecalin.

8. The composition of claim 1, wherein said aggregate diameter ranges from about 20 to about 100 micron.

9. The composition of claim 1, wherein said islet cells or precursors thereof are human in origin.

10. The composition of claim 1, wherein said islet cells or precursors thereof are engineered to express a protein of interest.

11. A method of increasing pancreatic islet cell mass; improving glucose homeostasis; or inhibiting, suppressing or treating diabetes in a subject, said method comprising administering to the subject the composition of claim 1.

12. The method of claim 11, wherein said subject is suffering from or predisposed to diabetes.

13. The method of claim 11, further comprising the step of administering to said subject a sulfonylurea, leptin, meglitinide, biguanide, thiazolidinedione, alpha-glucosidase inhibitor, or a combination thereof.

14. The method of claim 11, wherein said composition comprises islet cells or precursors thereof which are autologous, allogeneic, syngeneic or xenogeneic with respect to said subject.

15.-28. (canceled)

29. The method of claim 11, wherein said subject is insulin resistant or hypoinsulinemic.

30. A process for the preparation of an islet cell or precursor cell product encapsulated in a biocompatible matrix comprising a fluorocarbon emulsion, said process comprising:
a isolating and dispersing islet cells or precursors thereof from a pancreas of a subject or from a tissue or cell culture;
b in vitro culturing dispersed islet cells or precursors thereof obtained in (b) for a period of time sufficient to form aggregates of said islet cells or precursors thereof in culture; and
c encapsulating aggregates obtained in (b) within a matrix comprising an emulsion comprising a biocompatible polymer and a fluorocarbon.

31. The process of claim 30, wherein said biocompatible polymer comprises alginate.

32. The process of claim 31, wherein said alginate is at a final concentration of about 0.3 to about 4% w/v of said encapsulated islet cell or precursor cell product.

33. The process of claim 30, wherein said fluorocarbon is at a concentration of about 40 to about 85% w/v of said encapsulated islet cell or precursor cell product.

34. The process of claim 30, wherein said fluorocarbon comprises perfluorodecalin.

35. The process of claim 30, wherein said aggregate has a diameter of about 20 to about 100 micron.

36. The process of claim 30, wherein said islet cells or precursors thereof are human in origin.

37. The process of claim 30, further comprising the step of engineering said dispersed islet cells or precursors thereof to express a protein of interest.

38. The process of claim 30, wherein said encapsulating comprises extrusion of said matrix through a droplet generator.

39. The process of claim 30, wherein capsules of about 350 μm to 3 mm in diameter are formed.

40. A composition comprising at least one islet, islet fragment, islet cell or precursor thereof encapsulated in a matrix, wherein said matrix comprises a biocompatible polymer and a fluorocarbon, wherein said fluorocarbon:
a. is dispersed in said matrix; and
b. is at a concentration of about 40 to about 85% w/v of said composition.

41. The composition of claim 40, wherein said composition comprises a plurality of islets, islet fragments, islet cells or precursors thereof.

42. The composition of claim 40, wherein said biocompatible polymer comprises alginate.

43. The composition of claim 40, wherein said fluorocarbon comprises perfluorodecalin.

44. The composition of claim 40, wherein said at least one islet, islet fragment, islet cell or precursor thereof is human in origin.

45. The composition of claim 40, wherein said at least one islet, islet fragment, islet cell or precursor thereof is engineered to express a protein of interest.

46. A method of increasing pancreatic islet cell mass; improving glucose homeostasis; or inhibiting, suppressing or treating diabetes in a subject, said method comprising administering to the subject the composition of claim 40.

47. The method of claim 46, wherein said subject is suffering from or predisposed to diabetes.

48. The method of claim 46, further comprising the step of administering to said subject a sulfonylurea, leptin, meglitinide, biguanide, thiazolidinedione, alpha-glucosidase inhibitor, or a combination thereof.

49. The method of claim 46, wherein said composition comprises islet cells or precursors thereof which are autologous, allogeneic, syngeneic or xenogeneic with respect to said subject.

50.-63. (canceled)

64. The method of claim 52, wherein said subject is insulin resistant or hypoinsulinemic.

65. A method of increasing the viability, function, or combination thereof of islets, islet fragments, islet cells or precursors thereof said method comprising administering to the subject the composition of claim 40.

66. The method of claim 65, wherein said method reduces oxygen diffusion limitations in said islets, islet fragments, islet cells or precursors thereof, said matrix, or a combination thereof.

67. The composition of claim 1, wherein the islet cells or precursors thereof are derived from pancreas.

68. The composition of claim 1 wherein the islet cells or precursors thereof are derived from stem cells or progenitor cells.

69. The composition of claim 68 wherein the stem cells or progenitor cells are pluripotent precursor cells or multipotent precursor cells.

70. The composition of claim 1 wherein the islet cells or precursors thereof are derived from dispersed pancreatic islets.

71. The composition of claim 70 wherein the dispersed pancreatic islets comprise islet fragments.

72. The process of claim 30, wherein the islet cells or precursors thereof are derived from pancreas.

73. The process of claim 30 wherein the islet cells or precursors thereof are derived from stem cells or progenitor cells.

74. The process of claim 73 wherein the stem cells or progenitor cells are pluripotent precursor cells or multipotent precursor cells.

75. The process of claim 30 wherein the islet cells or precursors thereof are derived from dispersed pancreatic islets.

76. The process of claim 75 wherein the dispersed pancreatic islets comprise islet fragments.

77. The composition of claim 40, wherein the islet cell or precursor thereof is derived from pancreas.

78. The composition of claim 40 wherein the islet cell or precursor thereof is derived from a stem cell or a progenitor cell.

79. The composition of claim 78 wherein the stem cell or progenitor cell is a pluripotent precursor cell or a multipotent precursor cell.

80. The composition of claim 40 wherein the islet cell or precursor thereof is derived from a dispersed pancreatic islet.

81. The composition of claim 80 wherein the dispersed pancreatic islet comprises islet fragments.
